(19) United States
(12) Patent Application Publication
(10) Pub. No.: US 2022/0218963 A1
(43) Pub. Date: Jul. 14, 2022

(54) SURGICAL DRAIN REMOVAL DEVICE AND METHODS FOR USING SAME

(71) Applicant: RHODE ISLAND HOSPITAL, Providence, RI (US)

(72) Inventor: Albert TELFEIAN, Providence, RI (US)

(21) Appl. No.: 17/570,173

(22) Filed: Jan. 6, 2022

Related U.S. Application Data
(60) Provisional application No. 63/136,489, filed on Jan. 12, 2021.

(51) Int. Cl. A61M 27/00; A61B 17/32

(52) U.S. Cl. CPC A61M 27/002; A61B 2017/0023; A61B 17/32002

(57) ABSTRACT

Devices and methods for the percutaneous bedside removal of a surgical drain that is inadvertently sutured inside of a patient. The surgical drain removal device includes a hollow cylindrical body with opposed first and second open ends, the first end including cutting means. The first end and hollow cylindrical body fit over an end of a surgical drain that is partially sutured into a patient. The cutting means are moveable to cut a suture securing the surgical drain in a patient but do not cut the patient's skin. Methods and kits that use and include the surgical drain removal device are also disclosed. Use of the surgical drain removal devices, methods and kits alleviate the need for the surgeon and patient to return to the operating room to remove the drain in a subsequent surgical procedure.

### RELATED APPLICATION

This application claims priority to U.S. Provisional Patent Application Ser. No. 63/136,489, filed Jan. 12, 2021, the disclosure of which is hereby incorporated by reference in its entirety.

### FIELD OF THE INVENTION

The invention relates to surgical drains, and more particularly, to devices designed to facilitate a safe and effective removal of surgical drains from a patient's body, and methods for using same.

### BACKGROUND OF THE INVENTION

Surgical drains are used frequently in operations to allow fluid to drain out of a patient's body. Typically, these drains are made from flexible tubing that collects in a receptacle. Such receptacles include a bulb collection receptacle (e.g., a JP drain) or an accordion collection receptacle (e.g., a Hemovac drain), as both illustrated in FIG. 1. Surgical drains are placed near the end of the operation, and, after placement, the wound is closed.

One significant risk of placing a surgical drain is that the surgeon can, inadvertently, suture the drain into the patient (i.e., the surgical opening in the patient's body). This becomes evident when the surgeon (or other medical/surgical personnel) tries to remove the drain at the bedside. There is resistance noted if the drain has been sewn into the patient. Pulling too hard on the sewn-in drain can result in breaking the drain tubing, which then results in a retained foreign body in the patient. When the drain is sewn into the patient, it is typically necessary for the surgeon to return the patient to the operating room, open the incision, cut the suture that is preventing the drain from being removed, and remove the drain.

### SUMMARY OF THE INVENTION

The devices and methods of the present disclosure provides a safe and effective solution for percutaneously removing a surgical drain that is an inadvertently sewn (i.e., sutured) into a patient who has undergone an operation.

Various embodiments of a surgical drain removal device according to the present invention include a hollow cylindrical body with opposed first and second open ends, the first end including cutting means; wherein the first end and hollow cylindrical body are configured to fit over an end of a surgical drain; and wherein the cutting means are moveable to cut a suture securing the surgical drain in a patient.

According to another embodiment, a method for percutaneously removing a surgical drain sutured into a patient includes the steps of: (a) providing a surgical drain removal device including: a hollow cylindrical body with opposed first and second open ends, the first end including cutting means having at least one blunt edge on a first side and at least one sharp edge on a second side; wherein the first end and hollow cylindrical body are configured to fit over an end of a surgical drain; and wherein the cutting means are moveable to cut a suture securing the surgical drain in a patient; (b) fitting the first end and an adjacent portion of the hollow cylindrical body of the surgical drain removal device over the end of the surgical drain so that the cutting means is adjacent the patient's skin and the suture; (c) advancing the removal device towards the patient until the cutting means comes into contact with the patient's skin; (d) moving the first end and cutting means in a first direction so that the at least one blunt edge is moved into the patient skin without cutting into the patient skin, until resistance is met; (e) moving the first end and cutting means in a second direction opposite the first direction while applying tension so that the at least one sharp edge is advanced to cut through the suture securing the surgical drain within the patient; and (f) removing the surgical drain and suture from the patient.

According to a further embodiment, a kit for the percutaneous removal of a surgical drain that is sutured inside of a patient includes a plurality of surgical drain removal devices. Each surgical drain removal device includes a hollow cylindrical body with opposed first and second open ends, the first end including cutting means; wherein the first end and hollow cylindrical body are configured to fit over an end of a surgical drain; and wherein the cutting means are moveable to cut a suture securing the surgical drain in a patient. The plurality of surgical removal devices have different sizes to accommodate surgical drains of different sizes.

This Summary of the Invention is neither intended nor should it be construed as being representative of the full extent and scope of the present disclosure. The present disclosure is set forth in various levels of detail in the Summary of the Invention as well as in the attached drawings and the Detailed Description of the Invention, and no limitation as to the scope of the present disclosure is intended by either the inclusion or non-inclusion of elements, components, etc. in this Summary of the Invention. Additional aspects of the present disclosure will become more readily apparent from the Detailed Description, particularly when taken together with the drawings.

The above-described benefits, embodiments, and/or characterizations are not necessarily complete or exhaustive, and in particular, as to the patentable subject matter disclosed herein. Other benefits, embodiments, and/or characterizations of the present disclosure are possible utilizing, alone or in combination, as set forth above and/or described in the accompanying figures and/or in the description herein below. However, the Detailed Description of the Invention, the drawing figures, and the exemplary claim set forth herein, taken in conjunction with this Summary of the Invention, define the invention.

### BRIEF DESCRIPTION OF THE DRAWINGS

Embodiments of the invention are further described but are in no way limited by the following illustrations. The accompanying figures, which are incorporated in and constitute a part of the specification, illustrate preferred embodiments of the invention and, together with the description, serve to explain the objects, advantages, and principles of the invention. Embodiments of the invention are in no way limited by the following figures:

FIG. 1 shows two types of surgical drain/receptacle assemblies;

FIG. 2 is a top perspective exploded view of a surgical drain and a surgical drain removal device according to a first embodiment of the present invention;

FIG. 3 is a top perspective exploded view of the surgical drain removal device of FIG. 2 with a surgical drain extending from the body of a patient;

FIG. 4 is a top perspective view of the surgical drain removal device of FIG. 2 initially engaging a surgical drain extending from the body of a patient;

FIG. 5 is a top perspective view of the surgical drain removal device of FIG. 2 more extensively engaging a surgical drain extending from the body of a patient so as to cut a suture securing the surgical drain in the patient;

FIG. 6 is a detailed view of a portion of the surgical drain removal device of FIG. 2;

FIG. 7 is a schematic view of a surgical drain removal device according to a second embodiment of the present invention; and

FIG. 8 is a schematic view of a surgical drain removal device according to a third embodiment of the present invention.

### DETAILED DESCRIPTION OF THE INVENTION

Disclosed herein are devices for the percutaneous bedside removal of a surgical drain that is inadvertently sutured inside of a patient, thereby alleviating the need for the surgeon and patient to return to the operating room to remove the drain in a subsequent surgical procedure.

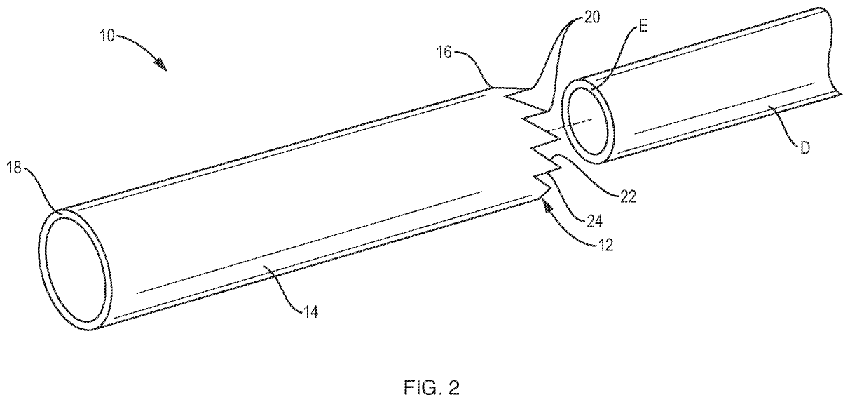

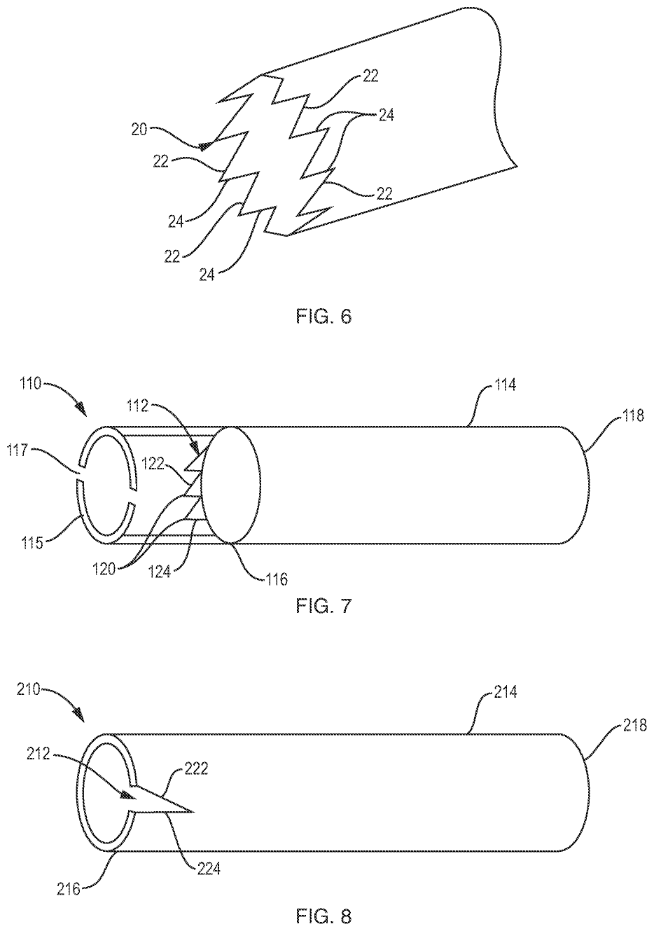

As illustrated in FIGS. 2-6, a first embodiment of a surgical drain removal device 10 of the present invention includes cutting means 12 and is sized to fit snugly over the open, distal end E of a standard surgical drain D. Cutting means 12 are moveable to cut a suture securing a surgical drain in a patient without injuring the patient, as further explained below. In the embodiment shown in FIGS. 2-6, cutting means 12 includes a crown reamer that is configured to cut a suture when moved in one direction, but not to cut a patient's skin when moved in another, opposite direction. In other embodiments, cutting means 12 includes other structures that are configured to cut suture when moved in one direction, but not to cut a patient's skin when moved in another, opposite direction (see FIGS. 7 and 8).

In various embodiments, the surgical drain removal device 10 is configured (e.g., sized) to accommodate surgical drains of different sizes (i.e., diameters). Such sizes include, as non-limiting examples, surgical drain diameters of 3 mm, 4 mm, 5 mm, 6 mm, and 7 mm. In other words, a removal device 10 according to embodiments of the invention is configured to accommodate a surgical drain having a 3 mm diameter, a 4 mm diameter, a 5 mm diameter, a 6 mm diameter or a 7 mm diameter. Surgical drawings having other sizes/diameters are also contemplated, where the removal device 10 may be configured accordingly.

In various embodiments, a kit is provided that includes multiple removal devices with crown reamers having different sizes (i.e., diameters), to accommodate surgical drains of different sizes. Such sizes include, as non-limiting examples, surgical drain diameters of 3 mm, 4 mm, 5 mm, 6 mm, and 7 mm.

In one embodiment, the surgical drain removal device 10 is disposable. In various embodiments, the removal device is made of one or more biologically acceptable materials. Non-limiting examples of such materials include ceramics, plastics and metals. Such metals include, as non-limiting examples, stainless steel, titanium, tungsten carbide and metal alloys.

Referring again to FIGS. 2-6, the surgical drain removal device 10 includes a hollow cylindrical body 14 with opposed first and second ends, 16, 18. The first end 16 includes the cutting means/crown reamer 12, which has a plurality of protruding teeth 20. As shown in FIGS. 2 and 4-6, each of the teeth 20 is formed to have a blunt, or first, edge 22 on a first side and a sharp, or second, edge 24 on a second side. The blunt edge 22 is configured to function as a leading edge and not cut a patient's skin, and the sharp edge 24 is configured to cut through a suture but not to cut the surgical drain D. The second end 18 of the removal device 10 has a smooth circular shape (i.e., without teeth or other cutting means).

The first end 16 and the hollow cylindrical body 14 (i.e., an adjacent portion thereof) are configured to fit over the open, distal end E and exterior portion (i.e., extending outside of a patient's body) D_{E} of a standard surgical drain D. Such a fit is snug and secure. In various embodiments, the fit between the first end 16 and hollow cylindrical body 14 and the open, distal end E is achieved by an interference fit, a friction fit or any other type of fitting that provides a snug and secure connection.

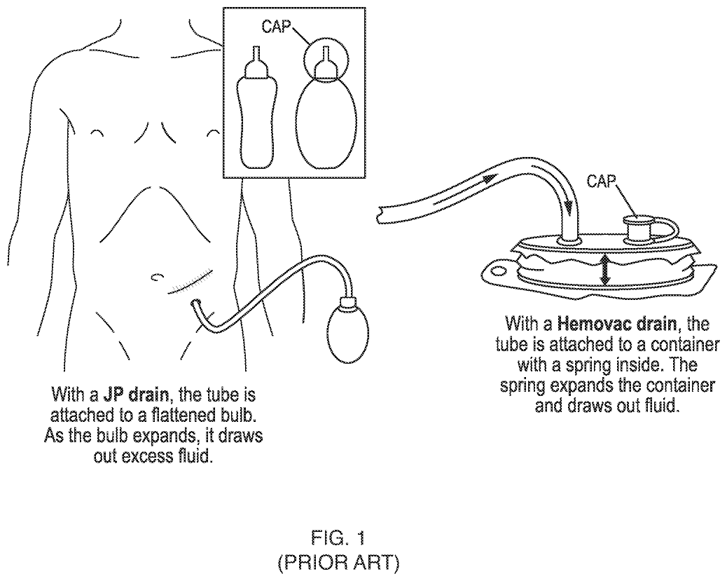

In operation, the area around the drain exit site of a patient is prepped in a standard fashion (see, e.g., FIG. 1). The drain receptacle is then removed, leaving the surgical drain D partially inside of a patient's body P (i.e., internal drain portion D_{I}) and extending partially outside of the patient's body P (i.e., external drain portion D_{E}). If a surgeon (or other medical/surgical personnel) inadvertently sews the internal drain portion D_{I} inside of the patient P (i.e., via a suture S sewn within the patient's body cavity), he or she may safely and efficiently remove the internal drain portion D_{I} from the patient P using the removal device 10 according to the following method.

The surgeon (or other medical/surgical personnel) obtains a removal device 10 from a hospital or surgical center's supply room. A set of differently sized removal devices 10 may be packaged together (e.g., in a kit) so that the surgeon (or other medical/surgical personnel) can select the correct size, based on the size of the surgical drain D.

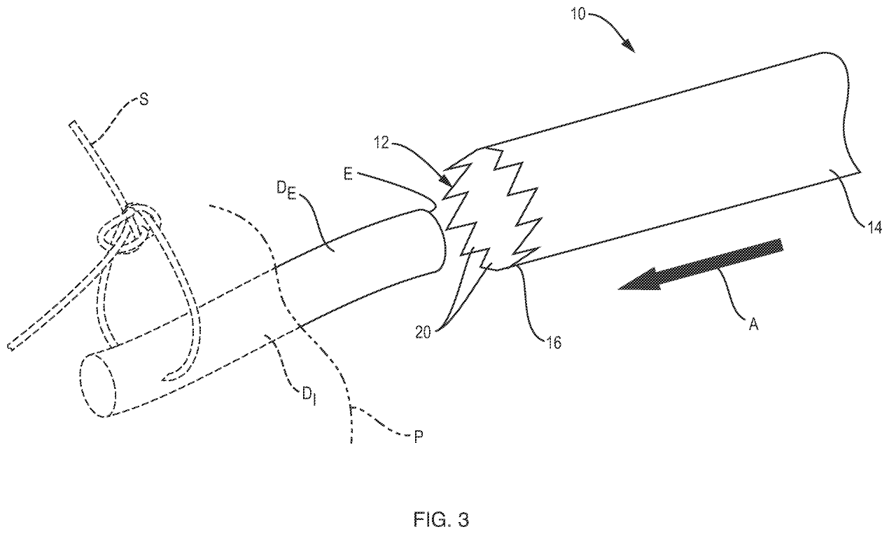

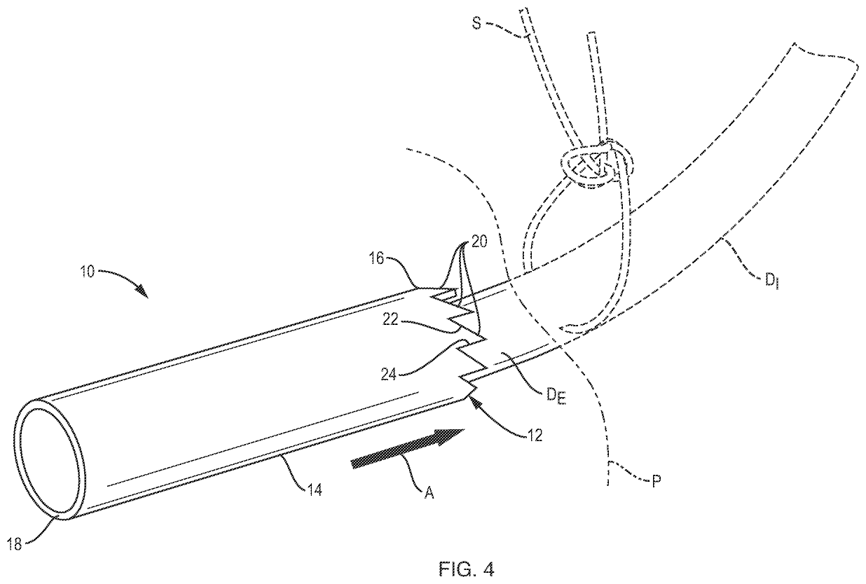

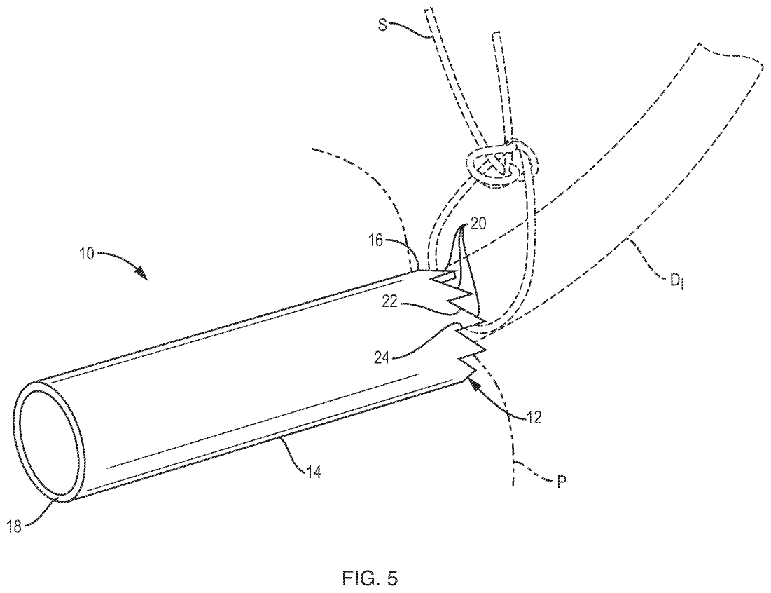

Once the correctly sized removal device 10 is provided, the first end 16 thereof with the cutting means/crown reamer 12 is placed over the open distal end E and external drain portion D_{E} of the drain D and the removal device 10 is advanced towards the patient P (i.e., distally from the surgeon (or other medical/surgical personnel)), as indicated by Arrow A in FIGS. 3 and 4. The cutting means/crown reamer 12 of the first end 16 is brought into contact with the patient P's skin and the crown reamer 12 is turned counterclockwise so that the first, blunt edges 22 of the teeth 20 are moved into the patient P's skin without cutting into the patient P's skin, until resistance is met (see FIG. 5). The crown reamer 12/first end 16 of the removal device 10 is then turned clockwise with a gentle tension applied, so that the second, sharp edges 24 of the teeth 20 are advanced to cut through the suture S securing the internal drain portion D_{I} within the patient P. This facilitates the easy and safe removal of the internal drain portion D_{I}, and the entire surgical drain D (e.g., still coupled to the removal device 10) from the patient P. The surgical drain D, removal device 10 and cut suture S can then be disposed of as medical waste.

In an alternate embodiment, the teeth 20 of the surgical drain removal device 10 are configured such that the removal device 10 is turned clockwise first, so that the first, blunt edges 22 of the teeth 20 move into the patient's skin without cutting into the patient's skin, until resistance is met. The crown reamer 12/first end 16 of this embodiment of the removal device 10 is then turned counterclockwise with a gentle tension applied, so that the second, sharp edges 24 of the teeth 20 are advanced to cut through the suture S.

A second embodiment of a surgical drain removal device 110 of the present invention is shown in FIG. 7. The removal device 110 includes a hollow cylindrical body 114 with opposed first and second ends, 116, 118. The first end 116 includes cutting means 112 having a plurality of protruding teeth 120 (i.e., a saw). Each of the teeth 120 is formed to have a blunt, or first, edge 122 on a first side and a sharp, or second, edge 124 on a second side. The blunt edge 122 functions as a leading edge, and the sharp edge 124 is configured to cut through a suture (not shown), with an otherwise generally similar structure and operation as the removal device 10 described above and illustrated in FIGS. 2-6.

The removal device 110 further includes a chamber 115 proximate the first end 116 and having a slot 117 that facilitates the capture of the suture into the chamber 115, followed by the end and external portion of the surgical drain. The cutting means 112 are internally housed within the chamber 115. Once captured in the chamber 115, the suture is cut by the second, sharp edge 124 of the cutting means 112.

A third embodiment of a removal device 210 of the present invention is shown in FIG. 8. The removal device 210 includes a hollow cylindrical body 214 with opposed first and second ends, 216, 218. The first end 216 includes cutting means 212 including a cut-out configured to have a blunt, or first, edge 222 on a first side and a sharp, or second, edge 224 on a second side. The blunt edge 222 functions as a leading edge, and the sharp edge 224 is configured to cut through a suture (not shown), with an otherwise generally similar structure and operation as the removal device 10 described above and illustrated in FIGS. 2-6.

The dimensions of the blunt edges 22, 122, 222 and sharp edges 24, 124, 224 may be suitably chosen in order to carry out the practice of the invention disclosed herewith. The exact configuration and geometry of the blunt and sharp edges providing the desired level of bluntness and sharpness can be determined by one skilled in the art without undue experimentation, and all such configurations are to be included within the scope of the present invention.

The removal devices of the present invention allow for the rapid bedside removal of a surgical drain which has been inadvertently sewn into a patient. Hospitals and surgical centers would benefit from keeping a supply of the removal devices in stock and using them with surgical drains, as doing so would prevent a return to the operating room for the surgical removal of the inadvertently sewn-in drains. These inadvertently sewn-in drains add unnecessary costs to the hospitals/surgical centers and expose the hospitals/surgical centers and the surgeon to enormous liability, given the surgical/medical complications that they may cause in the patient.

Modifications, additions, or omissions may be made to the systems, apparatuses, and methods described herein without departing from the scope of the disclosure. For example, the components of the systems and apparatuses may be integrated or separated. Moreover, the operations of the systems and apparatuses disclosed herein may be performed by more, fewer, or other components and the methods described may include more, fewer, or other steps. Additionally, steps may be performed in any suitable order. As used in this document, “each” refers to each member of a set or each member of a subset of a set.

Other embodiments of the invention will be apparent to those skilled in the art from consideration of the specification and practice of the invention disclosed herein. Although specific advantages have been enumerated above, various embodiments may include some, none, or all of the enumerated advantages. It is intended that the embodiments described above be considered as exemplary only, with a true scope and spirit of the invention being indicated by the appended claims. Moreover, none of the features disclosed in this specification should be construed as essential elements, and therefore, no disclosed features should be construed as being part of the claimed invention unless the features are specifically recited in the claims. In addition, it should be understood that any of the features disclosed on any particular embodiment may be incorporated in whole or in part on any of the other disclosed embodiments.

In any interpretation of the claims appended hereto, it is noted that no claims or claim elements are intended to invoke or be interpreted under 35 U.S.C. 112(f) unless the words “means for” or “step for” are explicitly used in the particular claim.

In general, any combination of disclosed features, components and methods described herein is possible. Steps of a method can be performed in any order that is physically possible.

All cited references are incorporated by reference herein.

Although embodiments have been disclosed, it is not desired to be limited thereby. Rather, the scope should be determined only by the appended claims.

While various embodiment of the present disclosure have been described in detail, it is apparent that modifications and alterations of those embodiments will occur to those skilled in the art. However, it is to be expressly understood that such modifications and alterations are within the scope and spirit of the present disclosure, as set forth in the following claims.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more embodiments for the purpose of streamlining the disclosure. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate preferred embodiment of the disclosure.

Moreover, though the present disclosure has included description of one or more embodiments and certain variations and modifications, other variations and modifications are within the scope of the disclosure, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

I claim:

1. A surgical drain removal device for the percutaneous removal of a surgical drain sutured inside of a patient, comprising:
a hollow cylindrical body with opposed first and second open ends, the first end including cutting means;
wherein the first end and hollow cylindrical body are configured to fit over an end of a surgical drain; and
wherein the cutting means are moveable to cut a suture securing the surgical drain in a patient.

2. The surgical drain removal device of claim 1, wherein the cutting means are moveable to cut a suture securing the surgical drain in a patient without injuring the patient or cutting the drain.

3. The surgical drain removal device of claim 1, wherein the cutting means includes a plurality of teeth.

4. The surgical drain removal device of claim 3, wherein each of the teeth includes at least one blunt edge on a first side and at least one sharp edge on a second side, the blunt edge being configured to not cut a patient's skin, and the sharp edge being configured to cut through a suture.

5. The surgical drain removal device of claim 3, wherein the cutting means further includes a crown reamer having the plurality of teeth thereon.

6. The surgical drain removal device of claim 3, further comprising a chamber proximate the first end and having a slot that facilitates the capture of the suture into the chamber, whereby the suture is positioned for cutting by the cutting means.

7. The surgical drain removal device of claim 1, wherein the cutting means includes a cut-out having a blunt edge on a first side and a sharp edge on a second side, the blunt edge being configured to not cut a patient's skin, and the sharp edge being configured to cut through a suture.

8. The surgical drain removal device of claim 1, wherein the fit between the hollow cylindrical body and the distal end of the surgical drain is selected from the group consisting of an interference fit, a friction fit, and any other type of fit that provides a secure connection.

9. The surgical drain removal device of claim 1, wherein the first end and hollow cylindrical body are configured to fit a surgical drain having a diameter selected from the group consisting of 3 mm, 4 mm, 5 mm, 6 mm, and 7 mm.

10. The surgical drain removal device of claim 1, wherein the surgical drain removal device is formed from a biologically acceptable material selected from the group consisting of ceramic, plastic and metal.

11. The surgical drain removal device of claim 10, wherein the metal is selected from a group consisting of stainless steel, titanium, tungsten carbide and metal alloys.

12. The surgical drain removal device of claim 1, wherein the surgical removal device is disposable.

13. A method for percutaneously removing a surgical drain sutured into a patient, comprising the steps of:
(a) providing a surgical drain removal device including:
a hollow cylindrical body with opposed first and second open ends, the first end including cutting means having at least one blunt edge on a first side and at least one sharp edge on a second side;
wherein the first end and hollow cylindrical body are configured to fit over an end of a surgical drain; and
wherein the cutting means are moveable to cut a suture securing the surgical drain in a patient;
(b) fitting the first end and an adjacent portion of the hollow cylindrical body of the surgical drain removal device over the end of the surgical drain so that the cutting means is adjacent the patient's skin and the suture;
(c) advancing the removal device towards the patient until the cutting means comes into contact with the patient's skin;
(d) moving the first end and cutting means in a first direction so that the at least one blunt edge is moved into the patient skin without cutting into the patient skin, until resistance is met;
(e) moving the first end and cutting means in a second direction opposite the first direction while applying tension so that the at least one sharp edge is advanced to cut through the suture securing the surgical drain within the patient; and
(f) removing the surgical drain and suture from the patient.

14. The method of claim 13, wherein the first direction is counterclockwise, and the second direction is clockwise.

15. The method of claim 13, wherein the first direction is clockwise, and the second direction is counterclockwise.

16. The method of claim 13, wherein the surgical drain removal device further includes a chamber proximate the first end and having a slot, the method further comprising the step of capturing the suture through the slot and into the chamber, whereby the suture is positioned for cutting by the cutting means.

17. The method of claim 13, wherein the fitting step is selected from the group consisting of interference fitting, friction fitting, and any other type of fitting that provides a secure connection.

18. The method of claim 13, further comprising the step of selecting the surgical drain removal device having a size corresponding to the surgical drain.

19. A kit for the percutaneous removal of a surgical drain that is sutured inside of a patient, comprising a plurality of surgical drain removal devices, wherein each surgical drain removal device includes a hollow cylindrical body with opposed first and second open ends, the first end including cutting means;
wherein the first end and hollow cylindrical body are configured to fit over an end of a surgical drain; and
wherein the cutting means are moveable to cut a suture securing the surgical drain in a patient; and
wherein the plurality of surgical drain removal devices have different sizes to accommodate surgical drains of different sizes.

20. The kit of claim 19, wherein the surgical drains of different sizes have diameters of 3 mm, 4 mm, 5 mm, 6 mm, and 7 mm.
